(19) United States
(12) Patent Application Publication
(10) Pub. No.: US 2003/0148350 A1
(43) Pub. Date: Aug. 7, 2003

(54) Method for determining telomere length and its use for assessing age

(76) Inventor: Peter Lahnert, Schwanebeck (DE)

Correspondence Address: JORDAN AND HAMBURG LLP, 122 EAST 42ND STREET, SUITE 4000, NEW YORK, NY, 10168, US

(21) Appl. No.: 10/316,599

(22) Filed: Jan. 29, 2003

(30) Foreign Application Priority Data
Dec. 15, 2001 (DE) 101 61 829.8-41

(51) Int. Cl. C12Q 1/68

(52) U.S. Cl. 435/6

(57) ABSTRACT

Age or expectation of life is assessed by determining the length of the telomeres by a blot method and, optionally, by subsequently hybridizing the DNA gel-electrophoretically separated and bound to the membrane obtained through the blot method by means of a nucleic acid probe complementary to the telomeric sequence, and subjecting the specimen DNA to a full restriction digestion containing the telomeric DNA prior to the gel-electrophoretic separation, wherein the separation of the digested DNA in the gel-electrophoresis underlying the blot method takes place at <5 V/cm and for the duration of at least 6 hours.

### DESCRIPTION

1. Field of Invention

This invention concerns a procedure for assessing age with the help of a tissue sample. Furthermore, this procedure, carried out in accordance with the invention, can most likely be used to determine statistical life expectancy of human beings.

2. Background of the Invention

No procedures have been described as yet within state-of-the-art technology, that could be used to determine the age or the average life expectancy of human beings.

In a textbook on Forensic Medicine (Arbab-Zadeh A., Prokop O., Reimann W., Rechtsmedizin für Ärzte, Juristen, Studierende und Kriminalisten [[ ](Forensic Medicine for Physicians, Jurists, Students and Criminalists[ ]]), 1. edition, Stuttgart, N.Y.: Gustav-Fischer-Verlag, 1977.) the following piece of information can be found: “Is this a child's blood or is it from an adult? At the moment this question can not be answered from lanes of blood.”

There is, however, a well established method for distinguishing the blood of an infant from that of an older child or an adult, which is checking the presence of foetal hemoglobin (Arbab-Zadeh A., supra).

But forensic medicine in particular would benefit greatly from a method to assess the age of a perpetrator—when profiling that person—with a tissue sample found at the site of the crime.

Inquiries with criminal prosecution and the police have also shown that there is no procedure yet to determine donor age from samples of blood or saliva. Both agencies express strong interest in such a procedure.

It would be yet another advantage, if a person's statistical life expectancy could be determined from a tissue sample. Because the shortening of the telomeres is regarded as the reason for natural aging (Fossel M D. Telomerase and the Aging Cell, JAMA (1998) 279, 1732-1735), people with long telomeres shot live longer than people with short telomeres. This could be valuable to insurance companies.

Tests have already been performed to measure telomere length (precisely: length of the terminal restriction fragment), proving that telomere length decreases continuously during in vivo aging (Hastie N. D., Dempster M., Dunlop M. G., Thompson A. M., Green D. G., Allshire R. C. Telomere reduction in human colorectal carcinoma and with aging. Nature (1990) 346, 866-868; Vaziri H., Schächter F., Uchida I., Wie L., Zhu X., Effros R., Cohen D., Harley C B., Loss of Telomeric DNA during Aging of Normal and Trisomy 21 Human Lymphocytes., Am. J. Hum. Genet. (1993) 52, 661-667).

The level of correlation between in vivo aging and telemore reduction, however, has not been sufficiently investigated. So, until the present invention, it has been impossible in practice to decide the age from a person's tissue sample.

Moreover, a test by Levy et al. has shown that there is no age specific telomere reduction (Levy T., Agoulnik I., Atkinson E. N., Tong X. W., Gause H. M., Hasenburg A., Ruimebaum I. B., Stickeler E., Möbus V. J., Kaplan A. L., Kieback D. G., Telomere Length in Human White Blood Cells Remains Constant with Age and is Shorter in Breast Cancer Patients, Anticancer Research (1998) 18, 1345-1350).

Due to these contradictory statements within the field, it has been completely unclear, if measuring telomere length could be utilized at all to determine age.

For the research papers mentioned above telomere length was established through telomere specific nucleic acid marked with ³²P.

There is also one non radioactive kit for assessing telomere length commercially available. It is the Roche Diagnostics GmbH, Telo TTAGGG Telomere Length Assay Kit, which uses a chemiluminescent substrate to detect telomere DNA. It is, however, a disadvantage that the corresponding procedure, according to the manufacturer, results in the lower and upper edges of the telomere smear being in different places according to the amount of DNA separated in the gel, after the gel electrophoresis is finished.

There is another publication (Gomez D. E., Tejera A. M., Olivero O. A., Irreversible Telomere Shortening by 3′-Azido-2′,3′--Dideoxythymidine (AZT) Treatment, Biochemical and Biophysical Research Communications (1998) 246, 107-110), where detection of telomere length was done with a color reaction. But no tissue samples were examined, nor was age determined. Additionally, no lower and upper edge were established, since telomere length was determined only once for each individual sample. (In order to realize there is a defined upper and lower edge at all, one has to do several telomere detection procedures with one DNA solution in different concentrations. Only then one can be certain to see the upper and lower edge.)

Moreover, blotting has apparently not worked properly, since the smears appear very pale when reproduced.

Also, no information is given about the voltage used and the running time of the gel.

There have been attempts by forensic institutions to infer age from telomere length (Jefferies J M C., Watson N D., Smith W E., Investigation af Donor Age Using Telomere Lengths from Simulated Biological Samples. Progress in Forensic Genetics (1999) 8, 27-29). Up to now this research has, however, not had tangible results.

In summary, this makes it clear that it is as yet not possible to determine the donor's age from a tissue sample.

Thus, in view of the above mentioned state-of-the-art technology, the task of providing a procedure to surmount this is the basis of this invention. The invention's procedure is intended to make it possible in particular, to determine a person's age and/or average life expectancy.

Further purposes that have not been explicitly mentioned, but are still reason for this invention, arise from and are closely connected to the current state of technology that has been mentioned and discussed.

These problems will be solved in an amazingly simple way through the preferred performance examples of the first claim and the claims related back to it.

### [DETAILIED] DETAILED DESCRIPTION

By determining telomere DNA length, it is possible to determine the sample donor's age and most likely the average life expectancy in a rather simple manner, by comparing it with ascertained samples according to the positioning of the signal on the southern blot.

For this, the telomere DNA length of a tissue sample is being determined with a southern blot procedure and ensuing hybridization of the DNA connected to the membrane, separated by gel electrophoresis, that has been obtained through the southern blot procedure with a nucleic acid probe that is complementary to the telomere sequence.

In this process, the sample DNA containing telomere DNA is subjected to a complete restriction digestion—before the gel electrophoresis separation—through a restriction enzyme, that has a specific identification point of 4-base pairs, and it is characterized as follows:

(a) the separation of the digested DNA in the gel electrophoresis, which is the basis of the southern blot procedure, is being done at <5 volt/cm and for a period of at least 6 hours,

(b) a marked oligonucleotide probe, complementary to the telomere sequence, is being used for the specific telomere DNA detection for the hybridization,

(c) the detection of the telomere DNA complex obtained from step (b) is being carried out with a color reaction and

(d) comparison values are being determined through application of known samples in the gel electrophoresis, which is the basis of the southern blot procedure.

Proceeding in accordance with the invention makes it possible, surprisingly, to increase the accuracy of measuring the (medium) telomere length so much, that age can be determined for some of the samples' donors.

Proceeding in accordance with the invention furthermore makes it possible to establish a lower and upper edge of the signal that has been obtained through the southern blot procedure, which can be used as an indication for determining length, apart from the medium telomere length.

In this procedure, as opposed to the methods used until now, the exact concentration of the DNA used is surprisingly not a decisive factor, because with the invention's method, as opposed to the traditional methods for determining telomere length, the lower and upper edge of the signal has an almost consistent position on the southern blot for a large range of DNA concentrations.

The southern blot procedure, very well known to experts, is used to transfer DNA from a gel, agarose or polyacrylamide for instance, after the gel electrophoresis separation, onto membranes, nylon membranes for instance, which are then accessible for other analytical processes.

For example, the DNA that is attached to the membrane can be hybridized with specific DNA probe molecules. The specificity of the hybridization reaction is being controlled through the choice of reaction conditions, in which no-stringent (high content of salt in the hybridization buffer, low temperature) or stringent hybridization conditions (low content of salt in the hybridization buffer, high temperature) are being used.

Normally the DNA probe molecules are being marked in some way. For example, the probe molecules could be marked through radioactivity, or carry a residue of biotin, or they could be connected to enzymes, antibodies or other [proteines] proteins and/or peptides.

The probe molecules that have been specifically hybridized to the DNA on the membrane are then being detected through an agent, which interacts in some way with the marking connected to the probe molecule.

In the case of a radioactive marking it is sufficient to do an auto radiograph, where an x-ray film is being blackened by the radioactive marking in the place, where the specific probe molecules are connected to the membrane. The signal obtained on the x-ray film can then be allocated to the DNA that has been separated by gel electrophoresis.

In other cases for instance, clear substrates are being added that can interact with enzymes and release a colored product, that may then be documented through photography.

All this is well known among experts, and this performance example as well as others of the southern blot procedure used in accordance with the invention can be found in numerous textbooks, of which only a few will be mentioned here: Sambrook J:, Fritsch E F., Maniatis T. (1989) Molecular Cloning: A Laboratory Manual, New York: Cold Spring Habour; Bertram S., Gassen H G. (1991), Gentechnische Methoden, eine Arbeitsanleitung für das molekularbiologische Labor [[ ](Methods in Genetic Engineering, an Operation Manual for the Molecular Biological Laboratory[ ]]), Stuttgart, Jena, N.Y.: Fischer G.; Cornel M. (2000), Der Experimentator: Molekularbiologie [[ ](The Experimentator; Molecular Biology[ ]]), Heidelberg, Berlin: Spektrum Acad. Verlag.

For the purposes of this invention, it is particularly important that the separation through gel electrophoresis of the sample DNA, which has been treated with the restriction enzyme, is being run slowly, that is with <5 volt/cm, and over a long period of time, that is >4 hours. Thus a good separation of the sample DNA is guaranteed.

Furthermore, the procedure should be carried out using color reactions for the detection of the specific complex of telomere DNA and probe DNA.

Especially when there is only very little sample material and thus little DNA, as is often the case in forensics, it makes sense to stretch out the color reaction as long as possible, i.e. several days or even weeks. With this concept the intensity of the coloring can be brought up to a sufficient level. Color reaction should be construed as follows within the scope of this invention: Detection of the telomere smears is being done trough a visible chemical substance. This detection is called chromogene detection.

In contrast to this, the detection of telomeres in the indirect detection process is being done with some sort of radiation, either radioactive (P 32) or non radioactive (chemiluminescence, Roche), whereby radiation always has to be documented (for example with an x-ray film).

It is therefore clearly understandable to the expert, that for marking the oligonucleotides it is favorable to use such systems suitable for creating a specific color reaction and also well known in the field. Examples of suitable dyes are 5-bromo-4-chloro-3-indolyl phosphate (BCIP) and nitroblue tetrazolium (NBT) for the alkaline phosphatase. For the horseradish peroxidase chloro naphthol is a suitable dye.

Also, processing should be done without vortex mixing, if possible, so the DNA is not exposed to strong shear forces unnessecarily. The largest part of the isolated DNA should have a molecule length of over 30,000 base pairs.

Moreover, it is presumably favorable to use high concentrations of the marked oligo- or polynucleotide, complementary to the telomere sequence, for example 1 μg/ml, which is roughly ten times of the [concentration] concentration commonly used.

Oligo- or polynucleotide means: >6 base pairs and include nucleic acid similar substances like PNA's. In a favored performance method of the present invention an agarose gel is used, which has an agarose concentration of 1.3-1.6% (w/w) in a lower section and 1-1.4% (w/w) in an upper section; the gel electrophoresis is to be run for at least 8 hours, with the lower section being the one that determines the size of the lower edge of the telomere smear, and the upper section being the one that determines the size of the upper edge of the telomere smear. It has been shown that the separation works even better then and the lower and upper edge of the signal can be defined even clearer.

In another favored performance method of this invention different amounts of the DNA to be examined (i.e. as 1 μl, 5 μl and 10 μl) are being applied next to one another, since especially in high DNA concentrations and long reaction periods of the detection reaction, even those parts of the lane, where no hybridization has taken place, could be colored. It could then be difficult to define the lower and upper edge of the smear.

The just mentioned coloring of parts of the lane, where there are no telomeres present, happens first in the lane where the largest amount of DNA was applied. As long as the upper and lower edge in all lanes have the same length, one can be sure not to have been fooled by this [artefact] artifact.

In accordance with the invention the procedure can be carried out with all human tissue samples, from which DNA can be isolated. This will preferably be samples of, saliva and/or blood, skin, hair. Samples of other tissues or secretions can be used as well. As mentioned above, in order to define age it is simplest to compare the sample with one being tested in the same procedure. In another equally favored performance method of this invention statistical data are being collected for all cohorts in question, in order to define a calibration line, which is the basis of determining age.

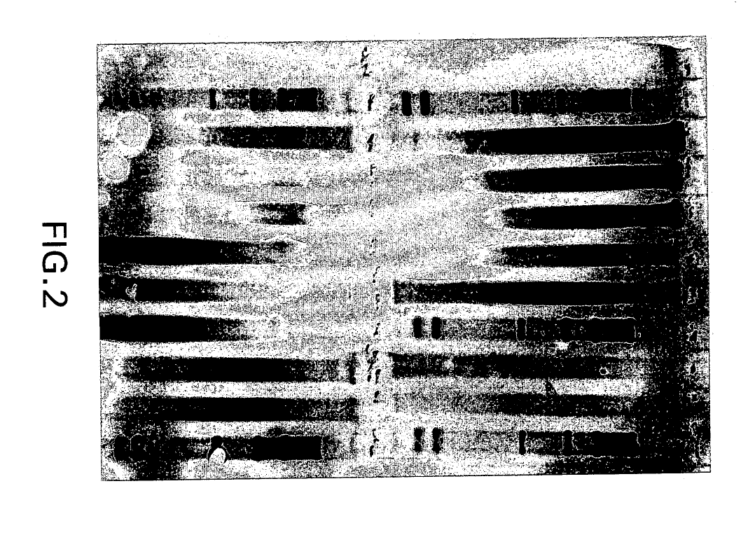

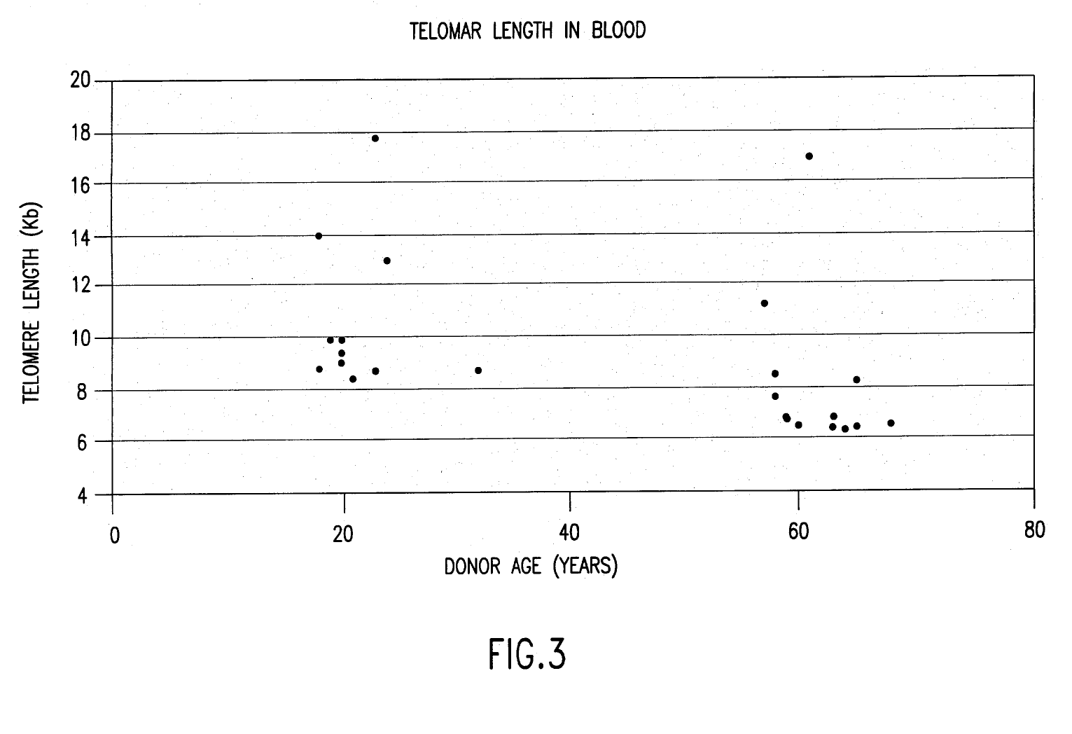

For defining a person's life expectancy especially, this is particularly convenient, since otherwise a very large number of comparison samples would have to be carried along in the test.

[Photo 1] FIG. 2 and [Photo 2 shows] FIG. 3 show the photographic evaluation of a southern blot procedure done in accordance with this invention. The specific test conditions are shown in example 1.

The following example serves to illustrate the invention, should however not be construed as a limitation.

### EXAMPLE 1

The genomic DNA collected from a saliva or blood specimen, 200 Al, was isolated by conventional methods (Sambrook, I.; Fritsch E F., Maniatis T. (1989), Molecular Cloning: A Laboatory Manual, New York: Cold Spring Habour; Bertam S., Gassen H G (1991), “Gentechnische Methoden, eine Arbeitsanleitung fur das molekularbiologische Labor”, Stuttgart, Jena, New York: Fischer G.).

(The specific method employed is not relevant; what alone is important is the fact that the DNA still is of a high molecular weight (the bulk of the un-cut DNA after isolation still has more than 30,000 base pairs, preferably 100,000 pairs, i.e. it was not exposed to excessive shearing forces). Accordingly, the specimens, during processing, should not be vortexed. Simple shaking or finger snapping will be adequate.

Thereafter, the DNA was completely cut by the restriction enzyme Alu I (Roche Diagnostics GmbH) having an identification sequence of four base pairs (genomic digestion). The restriction enzyme Alu I does not cut within the telomere as does the majority of enzymes. The cut DNA was electrophoretically separated in an agarose gel (1.3% w/w) at a 30 V voltage (corresponding to 1 V/cm) over 20 hours.

Then the gel (10×10 cm) was blotted equally by conventional methods, using a vacuum-blotter (Pharmacia) at 40 mbar:

After applying vacuum, 15 ml of a depurination solution (0.25 N HCl) was added to the surface of the gel. After 30 min the depurination solution was replaced by 15 ml of a denaturation solution (1.5 M NaCl; 0.5 M NaOH) for 30 min. Similarly, the denaturation solution was replaced by 15 ml of a neutralizing solution (1.0 M Tris; 2.0 M NaCl adjusted with HCl to pH 5.0) for 30 min.

Then the neutralizing solution was replaced by 20 ml 20×SSC for 60 min. From time to time an aliquot of 20×SSC was added in order to insure, that the gel be permanently covered by liquid. After completion of the transfer, the membrane was removed and washed in 10×SSC for 1 min. Then the DNA was fixed on the membrane by UV-radiation (125 mJ).

Then Prehybridization (for at least one hour) was performed in 20 ml of a prehybridization solution (5×SSC, blocking reagent (Roche) 1% (added from 10% stock solution), N-lauroylsarcosine 0.1%, SDS 0.02%) at 42° C. in a thermostat-controlled hybridization incubator.

After that the prehybridization solution was discarded and replaced by 15 ml of a hybridization solution (prehybridization solution and oligonucleotide probe (TTAGGG)₄ (1 μg/ml) labelled with digoxigenin). Before adding the same, the hybridization solution was preheated for 10 min at 90° C. but was not allowed to boil. After hybridization (overnight at 42° C.) the hybridization solution was frozen to be re-used in the next experiment.

The non-hybridized oligonucleotides freely contained in the hybridizing solution are removed by a number of washing steps: 1^{st} washing in 5×SSC (0.75 M NaCl, 0.0075M sodium citrate, pH-value 7.0) for 15 minutes at a temperature of 60° C.; 2^{nd} washing in 4×SSC for 15 minutes at 60° C.

The telomeres were then rendered visible by means of a labelling and detection reaction responding to the substance used for labelling purposes; preferably, the DIG DNA Labelling and Detection Kit from Roche Diagnostic (formerly Boehringer Mannheim).

As a DNA size standard (e.g. λ-Hind) normally is also separated during the gel electrophoresis, the telomere size could be determined by the distance covered.

As the telomere length is not identical for all cells within a cell population or even for all chromosomes within one cell, the telomeres do not appear as individual bands but rather as smears. The center of the smear is then assumed to be the (average) telomere length.

Now, the position of the telomere smears can be seen by the naked eye. It will thus already be possible to associate a large number of persons to the old or to the young group. However, in order to obtain precise molecular weights the telomere smears will have to be analyzed by a densidometer or a scanner.

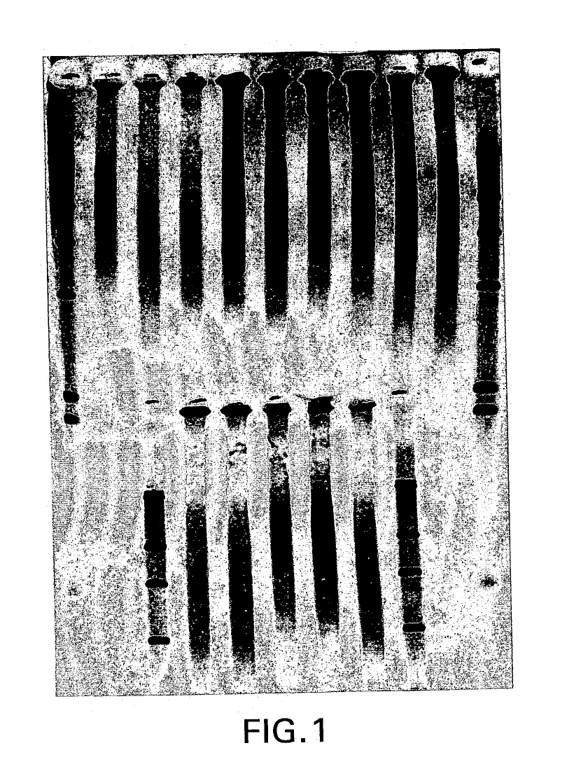

The Image Station 440 cf from Kodak in combination with the 1D Image Analysis Software likewise from Kodak, can be suitably used. Moreover, it can be useful to take a photo of the membrane and to scan the same. In particular, telomere smears of a low intensity can be analyzed more accurately in this way.

[Diagram 1] FIG. 1 shows the exact results for all blood specimens and shows the superiority of my method.

TABLE 1
([to photo] for FIG. 1)

| lane (top) | 1 | 2 | 3 | 4 | 5 | 6 | 7 | 8 | 9 | 10 | 11 |
|---|---|---|---|---|---|---|---|---|---|---|---|
| Age | λ- | 18 | 64 | 60 | 18 | 19 | 20 | 23 | 32 | 65 | λ |
| (years) | Hind | | | | | | | | | | Hind |
| Average | | 14.000 | 6.300 | 6.500 | 8.800 | 9.900 | 9.900 | 8.700 | 8.700 | 8.200 | |
| telomere | | | | | | | | | | | |
| length | | | | | | | | | | | |
| (bp) | | | | | | | | | | | |

| lane (below) | 1 | 2 | 3 | 4 | 5 | 6 | 7 |
|---|---|---|---|---|---|---|---|
| Age (years) | λ-Hind | 65 | 59 | 57 | 24 | 63 | λ-Hind |
| Average telomere length (bp) | | 6.400 | 6.800 | 11.20 | 13.00 | 6.800 | |
| Top border (bp) | | 25.800 | 21.30 | 25.20 | 30.60 | 27.90 | |
| Lower border (bp) | | 2.900 | 2.900 | 4.000 | 4.000 | 3.300 | |

Unfortunately, no upper or lower borders could be determined for the upper series as the colour of the background of the lane is too intensive. The only person that would be wrongly associated in this test would be the one in lane 4, bottom. It has relatively long telomeres although that person is already 57 years of age.

TABLE 2
([to photo[ for FIG. 2)

| | 1 | 2 | 3 | 4 | 5 | 6 | 7 | 8 | 9 | 10 |
|---|---|---|---|---|---|---|---|---|---|---|
| lane (top | | | | | | | | | | |
| Amout | λ- | 10 μl | 5 μl | 2μl | | | | | | λ-Hind |
| Average | | 8.500 | 8.500 | 7.100 | | | | | | |
| telomere length | | | | | | | | | | |
| (bp) | | | | | | | | | | |
| Top border | | 30.000 | 31000 | 30.000 | | | | | | |
| (bp) | | | | | | | | | | |
| Lower border | | 3.900 | 4.000 | 4.000 | | | | | | |
| (bp) | | | | | | | | | | |
| lane (below) | | | | | | | | | | |
| cell line | | | | | MM6 | MM6 | MM6 | U87 | U87 | |
| Amout | λ- | | | | 10 μl | 2 μl | 5 μl | 10 μl | 5 μl | λ-Hind |
| | Hind | | | | | | | | | |

In lanes 2, 3 and 4, top, different quantities of one and the same DNA preparation from blood (approximately 1 μg/μl DNA) were applied. It can be clearly seen that the upper and the lower border of the telomere smear, in all three lanes, have almost identical values.

Similarly, lanes 5 through 9 clearly reveal in respect of the telomere smears recovered from cell cultures that the smears have the same sizes. In view of the intensive background colouring, a numerical determination will involve substantial inaccuracies.

I claim:

1. A method of assessing the age or expectation of life wherein the length of the telomeres is determined by a blot method and, optionally, by subsequently hybridizing the DNA gel-electrophoretically separated and bound to the membrane obtained through the blot method by means of a nucleic acid probe complementary to the telomeric sequence, and wherein the specimen DNA containing the telomeric DNA prior to the gel-electrophoretic separation is subjected to a full restriction digestion, characterized in that the separation of the digested DNA in the gel-electrophoresis underlying the blot method takes place at
(a) <5 V/cm, and
(b) for the duration of at least 6 hours.

2. A method according to claim 1, wherein the complex composed of telomere DNA and probe nucleic acid is rendered visible by a colour reaction.

3. A method according to claim 1, wherein the concentration of the gel is higher than 1.0%.

4. A method for assessing the age according to claim 1, characterised in that by the comparison with a telomeric DNA obtained from an individual whose age is known, the age of the individual is determined from whom the tissue specimen has been collected.

5. A method according to claims 1, using an agarose gel which, in a lower section, is of a concentration of between 1.20% and 2.00% (w/w) and, in an upper section, is of a concentration of between 0.9% and 1.4%, and wherein the gel-electrophoresis is carried out for at least 6 hours, with the bottom section being the one in which the bottom border of the telomeric smear is precisely determined and the upper section being the one in which the upper border is determined.

6. A method according to claim 1, characterized in that a stable bottom border and a stable upper border of the TRF smear are detected by the combination of colour reaction, extended run and low electric voltage.

7. A method according to claim 1, characterized in that different quantities (e.g. 1 μl; 5 μl or 10 μl) of the DNA to be tested are applied in parallel.

8. A method according to claim 2, characterized in that the colour reaction is extended over several days or even months.

9. A method according to claim 1, characterized in that the telomeres are labelled directly before the restriction digestion (e.g. terminal transferase).

10. A method of estimating the average expectation of life of an individual on the basis of a tissue specimen or a secretion specimen, wherein the length of the telomeres of the DNA from the tissue or secretion specimen is determined by means of the method according to claim 1, wherein the secretion or tissue specimen, preferably, is a blood or saliva specimen.

11. A method according to claim 9, characterized in that, by the comparison with a telomeric DNA separation in the same test from a multiplicity of individuals that have died at a known age, the mean life expectancy of the person is determined from whom the tissue specimen has been taken.

12. A method according to claim 9, wherein the telomeric lengths are compared to a previously determined calibration standard and wherein no comparing specimens are used.
